(19) United States
(12) Patent Application Publication
(10) Pub. No.: US 2020/0090815 A1
(43) Pub. Date: Mar. 19, 2020

(54) A SYSTEM AND METHOD FOR SELECTING A PATIENT POPULATION FOR SPINAL REPAIR USING A MEASURABLE OUTCOME PREDICTIVE METRIC OF THE SPINE

(71) Applicant: Medical Metrics, Inc., Houston, TX (US)

(72) Inventor: John A. Hipp, Houston, TX (US)

(21) Appl. No.: 16/472,874

(22) PCT Filed: Dec. 21, 2017
(86) PCT No.: PCT/US17/67920

§ 371 Date: Jun. 22, 2019

Related U.S. Application Data
(60) Provisional application No. 62/438,328, filed on Dec. 22, 2016.

(51) Int. Cl. G16H 50/70; G16H 10/60; G16H 20/10; G16H 20/30; G16H 20/40; G16H 20/90

(52) U.S. Cl. CPC G16H 50/70; G16H 10/60; G16H 20/90; G16H 20/30; G16H 20/40; G16H 20/10

(57) ABSTRACT

The present disclosure describes systems and methods for determining whether a particular treatment would provide an extended benefit or a detriment to a subpopulation of patients. The system and methods provide a simple pictorial representation of patient benefits using an outcome predictive metric to see if any subpopulation produces significantly better results than the average or significantly worse results than the average.

The present disclosure describes systems and methods for determining whether a particular treatment provides an extended benefit or a detriment to any subpopulation of patients. More particularly, the system and methods provide a simple pictorial representation of patient benefits using an outcome predictive metric to see if any subpopulation produces significantly better results or significantly worse results than the average patient population. The pictorial representation, usually graphical, subsects the data using an increasing threshold or a decreasing threshold to look at subpopulations that might be better or worse candidates than the general population for receiving the particular treatment. Still more particularly, the disclosure describes a method for obtaining a quantitative metric for spinal motion (an outcome predictive metric) and obtaining outcome data for a particular spinal treatment, e.g., lumbar fusion, and plotting the patient success rates as a function of the metric for spinal motion. This graphical representation can provide physicians with a simple evaluation tool to assist them in determining the best treatments and procedures on a per patient basis.

### BACKGROUND

Predictive medicine, i.e., using mathematical correlations to determine the likelihood of contracting a disease, the best treatment options, life expectancies for certain diseases, etc., is a rapidly growing area. Predictive medicine will continue to push us toward a standard of care with more personalized medicine, since these predictions will be based upon patient specific criteria. While the full impact of predictive medicine is some way off, there are methods and systems that today can be used to personalize patient relevant information to assist physicians in making better treatment decisions. One such method and system for providing this type of personalized medical analysis is described.

According to one embodiment, the method and system as described look at specific treatments in the area of spinal treatment. Low back pain is one of the most common musculoskeletal complaints requiring medical attention. Low back pain is one of the primary reasons given for disability or time away from a job. Given the significant economic impact that spinal disorders cause, Medical Metrics, the assignee of the instant application, developed a reliable method for measuring and reporting spinal instability that is described in U.S. Pat. No. 9,265,463 (the '463 patent), which is incorporated herein by reference in its entirety. The disclosed method is reliable, relatively inexpensive, only requires information that is readily obtainable in a physician's office, easy to understand, and can be measured in patients regardless of their effort, since people with back pain often make less effort than may be required to diagnose spinal instability using other known methods.

The '463 patent describes a method for calculating a spinal translation per degree of rotation (TPDR) from diagnostic images that are independent of vertebrae size and do not require knowing the magnification of the image. The described method for calculating TPDR allows direct comparison of a symptomatic population to an asymptomatic population. The method generally includes, obtaining an x-ray film of first and second vertebrae in a first position; obtaining an x-ray film of the same first and second vertebrae in second position; marking the position of at least one landmark on the first vertebra on the x-ray film of the first position; superimposing the two x-ray films and aligning the first vertebra and determining the rotation and translation required to align the second vertebra in the two x-ray films; measuring the degree of vertebral rotation between the first and second vertebrae in the first and second positions; measuring the translation of the landmark on the first vertebra between the first and second positions while holding the second vertebra fixed; normalizing the intervertebral translation measurement based upon a vertebral dimension; dividing the normalized intervertebral translation by the intervertebral rotation to give a TPDR. The TPDR is standardized to the same vertebrae pair in an asymptomatic population to generate a stability metric. The stability metric is based upon an asymptomatic spine registering substantially zero and instability being displayed as a positive or negative deviation from the asymptomatic “normal” population. More specifically, the stability metric is reported as a number of standard deviations from normal, where normal refers to a level appropriate value for an asymptomatic population. This prior art approach to defining spinal instability has all the criteria for use in clinical diagnosis, but more importantly, this stability metric has good correlation with spinal treatment outcomes.

According to another embodiment, the disclosure describes a method and system for using the correlation between any outcome predictive metric and actual patient outcomes to ascertain whether a particular treatment is better or worse for certain sub-populations of patients. According to one embodiment, the invention includes a method for determining whether a particular treatment would provide an extended benefit or a detriment to a subpopulation of patients using an outcome predictive metric comprising, obtaining information regarding the clinical outcomes of a plurality of patients for a particular medical treatment, determining at least one quantitative metric that has an outcome predictive correlation with the disease condition being treated, plotting the percentage of people with beneficial outcomes as a function of the at least one outcome predictive metric, determining from this plot the baseline of beneficial outcomes by averaging the values of the quantitative metric over the ranges that are least variable, calculating the 95% confidence interval for the baseline beneficial outcomes, re-plotting the percentage of patients with beneficial outcomes using an increasing threshold and using a decreasing threshold, and determining if one or more sub-populations have a benefit or detriment that is outside the 95% confidence interval for the baseline of beneficial outcomes.

In yet another embodiment, the present disclosure describes a method for determining whether a spinal treatment would provide an extended benefit or a detriment to a subpopulation of patients using a stability metric comprising, obtaining an x-ray film of a first and a second vertebrae in a first position, obtaining an x-ray film of a first and second vertebrae in a second position, determining the rotation and translation required to align the second vertebra, measuring the degree of vertebral rotation between the first and second vertebrae in the first and second positions and calculating a TPDR, multiplying the TPDR by a standardizing factor specific to the first and second vertebrae to generate a stability metric, obtaining information regarding the clinical outcomes of a plurality of patients from a particular spinal treatment, plotting the percentage of patients with beneficial outcomes as a function of the stability metric using an increasing threshold and using a decreasing threshold, and determining if one or more sub-populations have a benefit or detriment that is outside the baseline of beneficial outcomes.

A better understanding of the various disclosed system and method embodiments can be obtained when the following detailed description is considered in conjunction with the drawings.

### BRIEF DESCRIPTION OF THE DRAWINGS

FIG. 1 is a bar graphical representation of the percentage of patients that had an ODI benefit of greater than 15 at twenty four months after Lumbar Fusion as a function of their pre-surgical QSI Stability Factor.

FIG. 2 is a graphical representation of the increasing threshold and decreasing threshold for patients that had an ODI benefit of greater than 15 at twenty four months after Lumbar Fusion as a function of their pre-surgical Intervertebral Rotation.

FIG. 3 is a graphical representation of the increasing threshold and decreasing threshold for patients that had an ODI benefit of greater than 15 at twenty four months after Lumbar Fusion as a function of their pre-surgical QSI Stability Factor.

FIG. 4 is a different graphical representation of the percentage of patients that had an ODI benefit of greater than 15 at twenty four months after Lumbar Fusion as a function of their pre-surgical QSI Stability Factor.

FIG. 5 is a graphical representation of the percentage of patients that had an ODI benefit of greater than 15 at twenty four months after Lumbar Disc Arthoplasty as a function of QSI stability factor.

FIG. 6 is a graphical representation of the percentage of patients that had an ODI benefit of greater than 15 at twenty four months after Lumbar Disc Arthoplasty as a function of their pre-surgical Standardized Disc Height.

FIG. 7 is a graphical representation of the percentage of patients that had an ODI benefit of greater than 15 at twenty four months after Cervical Disc Arthoplasty as a function of their pre-surgical QSI Stability Factor.

The drawing figures are not necessarily to scale. Certain features of the embodiments may be shown exaggerated in scale or in somewhat schematic form and some details of conventional elements may not be shown in the interest of clarity and conciseness.

### DESCRIPTION

The following discussion is directed to various embodiments of the invention. Although one or more of these embodiments may be preferred, the embodiments disclosed should not be interpreted, or otherwise used, as limiting the scope of the disclosure, including the claims. It is to be fully recognized that the different teachings of the embodiments discussed below may be employed separately or in any suitable combination to produce desired results. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not structure or function.

In the following discussion and in the claims, the terms “including,” “comprising,” and “is” are used in an open-ended fashion, and thus should be interpreted to mean “including, but not limited to.”

As used herein, the terms “spinal stability” and “spinal instability” are understood to be interchangeable except where specifically indicated as different or where one of ordinary skill in the art would understand them to be different and refer to the deviations in the movement of the spine in a symptomatic patient when compared to an asymptomatic population.

As used herein, the terms “intervertebral translation” and “translation” are understood to be interchangeable except where specifically indicated as different or where one of ordinary skill in the art would understand them to be different and refer to the lateral or gliding movement of a vertebra. Translation rarely occurs by itself, but often accompanies other movements of the vertebrae.

As used herein, the terms “intervertebral rotation” and “rotation” are understood to be interchangeable except where specifically indicated as different or where one of ordinary skill in the art would understand them to be different and refer to the motion of a vertebra around an axis.

As used herein, the terms “standardized TPDR” and “stability metric” are understood to be interchangeable except where specifically indicated as different or where one of ordinary skill in the art would understand them to be different and refer to the TPDR that has been calculated using a normalized translation value and which has been multiplied by a standardizing formula.

As used herein an “increasing threshold” refers to the subsecting of data according to a series of increasing thresholds. Consider each unit of the outcome predictive metric as a threshold. For the increasing threshold, one would begin the plot at the lowest measured outcome predictive metric and would then eliminate data at each new threshold. So, by way of example, if the lowest value for the outcome predictive metric is −3, then the first point at −3 would include all patients having a metric above −3 (which includes all patients as this is the lowest measured metric). Then at −2, one would plot the outcome but only for those patients having a metric of −2 or above. Likewise, at −1, one would plot the outcome, but only for those patients having a metric value of −1 or greater. This would continue until the highest measured outcome predictive metric is reached.

As used herein a “decreasing threshold” also refers to the subsecting of data, but this time, according to a series of decreasing thresholds. Consider each unit of the outcome predictive metric as a threshold. For the decreasing threshold, one would begin the plot at the highest measured outcome predictive metric and would then eliminate data at each new threshold. So, by way of example, if the highest value for the outcome predictive metric is 4, then the first point at 4, would include all patients below 4 (which includes all patients as this is the highest measured metric). Then at 3, one would plot the outcome but only for those patients having a metric of 3 or below. Likewise, at 2, one would plot the outcome, but only for those patients having a metric value of 2 or less. This would continue until the lowest measured outcome predictive metric is reached.

As used herein, the term Standardized Disc Height refers to the number of standard deviations from the average disc height found in radiographically normal levels in asymptomatic volunteers.

As used herein, QSI is a Quantitative Stability Index and refers to the stability metric that was described in the '463 patent. QSI is calculated using lumbar flexion and extension radiographs.

As used herein ODI refers to Owestry Disability Index. ODI is an index derived from a patient questionnaire on low back pain. The ODI questionnaire was first published by Jeremy Fairbank et al. in 1980. ODI has become a standard metric for low back pain.

The method and system as described are a new way of looking at outcome predictive metrics and determining whether a particular subpopulation of patients within that metric are better or worse candidates for use of the treatment method or drug, or surgery being considered. More particularly, the method and system develop pictorial representations based upon actual patient outcomes that allow a physician to easily look at the representation and determine whether the treatment under consideration is particularly favorable for patients exhibiting particular metrics. Still more particularly, the method and system use a pair of calculations including an increasing threshold and a decreasing threshold to determine if any subpopulation within the metric would have an enhanced chance of obtaining a favorable outcome or whether any subpopulation would have a diminished chance of obtaining a favorable outcome.

The method is carried out by first obtaining information regarding the clinical outcomes of a plurality of patients for a particular medical treatment. This information can be developed independently, can be ascertained from clinical trials or from reported study data. The information will need to include both outcomes for the particular disease state as well as pre-treatment metric measurements that have some correlation to the outcomes. As used herein beneficial outcomes are any measured outcome that the physician or lead investigator believes to be of interest. For example, the physician may want to look at patients having achieved a particular result, e.g., an ODI of greater than 15 at 24 months, as shown in the FIGS. The ODI of greater than 15 at 24 months is the beneficial outcome. Another beneficial outcome that might be measured in the same patient population is an ODI of greater than 15, at 12 months. The technique as described herein can be used to evaluate any treatment method, regardless of the medical area, in which a measured beneficial outcome has a measured pre-treatment outcome predictive metric. The method and pictorial representation as described herein can be used to provide useful information to physicians by clearly representing the efficacy of a particular treatment in different subpopulations of patients.

When developing and evaluating beneficial outcomes, the pre-treatment data collection will need to include measurement of the outcome predictive metric that one wishes to use for evaluation purposes. In the event that such was not the design of the original data collection, measured pre-treatment metrics can be evaluated to determine if any of them have result correlation.

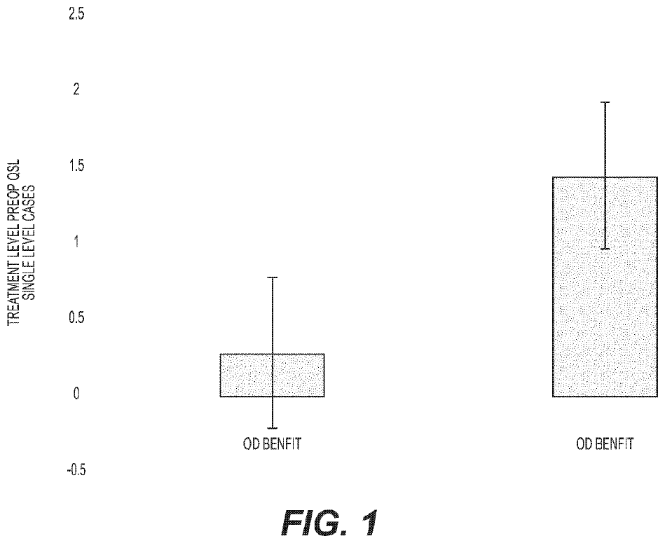

To determine whether a pre-treatment metric is outcome predictive, one would plot on one axis of a graph different potential threshold levels of the metric, and on the other axis the proportion of patients achieving a benefit or the average value of an outcome metric, both after excluding all subjects above a specific threshold and again after excluding all subjects below the threshold. The resulting plot will show little variability as a function of the specific threshold if the metric has no effect on outcomes. Alternatively, the plot will show a range or ranges of the metric where the treatment effect was either enhanced above a baseline or reduced below a baseline if the metrics has an effect on outcomes. So, for example, if a measured metric is on a scale of 0 to 5, and you wish to know whether it is outcome predictive, a threshold would be selected, e.g., three, and one would plot the percentage of the population receiving a beneficial outcome is substantially the same. An exemplary plot can be found in FIG. 1.

FIG. 1 is a graphical representation of QSI for postero-lateral fusion patients that had a beneficial outcome (in this graph an ODI of greater than 15) versus those who did not. This graph shows that there is a correlation between the QSI and the benefit achieved. If there were no correlation, you would expect the two bars to be similar, i.e., the good and bad results would be found at all QSI levels.

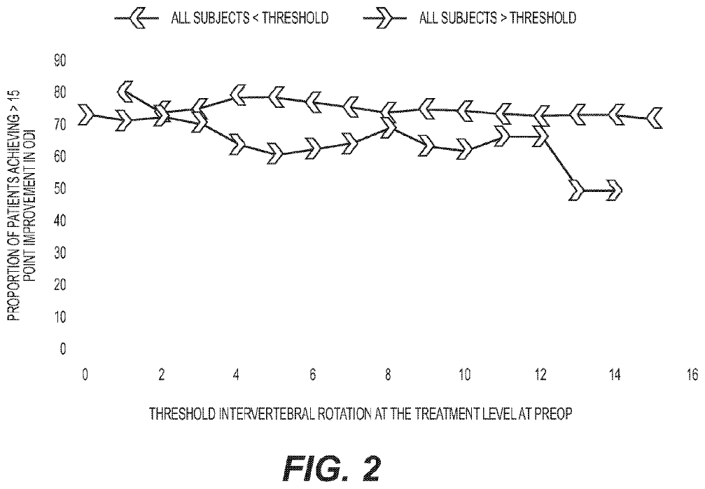

If a measured metric has little or no effect on treatment outcomes, this approach to identifying a subpopulation for enhanced or reduced treatment benefit is not as helpful. A graphical representation of a non-outcome predictive metric can be seen in FIG. 2. This graph is based upon many peer-reviewed publications that use the magnitude of intervertebral rotation to identify levels of vertebrae that were considered unstable and therefore were candidates for fusion. Many papers have used a threshold level of greater than 10 degrees to justify fusion. The graph in FIG. 2 shows the enhanced/reduced treatment benefit plot for intervertebral rotation. Including all of the patients in the study, 72.3% had greater than 15 point improvement. As you can see, there is not much variability in the proportion of patients achieving the clinical benefit, except for rotations greater than or equal to 13 degrees, where there is actually a reduced treatment benefit. This graph supports that the criteria so commonly used may not correlate to treatment outcomes as well as expected.

If the outcome, when including patients either above or below a threshold level of the metrics, does not change substantially no matter what threshold is chosen, than it can be said that the metric is not predictive of outcome (at least not predictive of the specific outcome being assessed).

Another way to determine whether a metric is outcome predictive could be to look at a “substantial” change as being a change greater than what a peer-reviewed publication describes as a clinically meaningful change. Clinically meaningful change according to some studies is a change in pain measured on a visual-analog-scale (VAS) that is greater than 2.5 out of 10. If we than looked at a graph, it shows the mean value for the whole study population, and defined an upper and lower limit as 2.5 units on either side, than that could be used to identify whether the average value for patients above or below a threshold level of the outcome predictive metric (OPM) is outside of the limits. If it is above the upper limit or below the lower limit than the OPM is predictive of outcomes.

Once the beneficial outcome has been decided and the outcome predictive metric has been selected, the method begins by plotting the percentage of people with the beneficial outcomes as a function of the at least one outcome predictive metric. This plot will be used to determine the baseline benefit (similar to average benefit) that the particular treatment achieved for the average population. The baseline is calculated by looking at the plot and determining the range or ranges of the outcome predictive metric that is least variable. The baseline should reflect the most common benefit that was achieved by the population. This is generally seen in the areas of the graph where outcomes are more consistent, i.e., the points are less variable. The baseline is calculated as the average benefit over the graph segment that has the least variability. For one example of the “least variable segment, See, FIG. 1, where the baseline would be calculated based upon the graphed averages on the increasing threshold line (right facing arrows) from −3 to about 0.5.

Once the baseline is calculated, one will need to determine the range around the baseline that defines the expected benefits of the total population, for example, a 95% confidence interval for the baseline. This confidence interval will yield the range of what would be the expected benefit for the average population. According to one embodiment, a plot does not have to be created to calculate the baseline or the 95% confidence interval the calculation of the 95% confidence interval as either may be calculated using an appropriate program on any computer or hand held device.

The range around the baseline that defines the expected outcomes that would appear in the total population can be calculated not as a confidence interval, but alternatively may be set by using the minimum and maximum values for the region chosen to define the baseline. These minimum and maximum values would set the values for which an extended result or a diminished result would begin. As another alternative, one could use data from publications describing the minimum clinically important difference (MCID) for the procedure in question and then identify the baseline and then use baseline value plus the MCID to set the top of the range and the baseline minus the MCID for the lower end of the range. As the methods and system as described, are developing information on the subpopulations that do not meet the average expectation or that exceed the average expectation, this range will be the reference range to which subpopulation data is compared.

The original data used to determine the baseline will then be re-plotted (or initially plotted if the baseline/confidence interval was merely calculated). The plot(s) will look at the percentage of patients with beneficial outcomes using an increasing threshold and using a decreasing threshold. According to one embodiment, the increasing threshold and the decreasing threshold will be plotted on the same graph. From these two graphical representations, one can easily see whether one or more subpopulations have a benefit or detriment that is outside the 95% confidence interval for the baseline of beneficial outcomes.

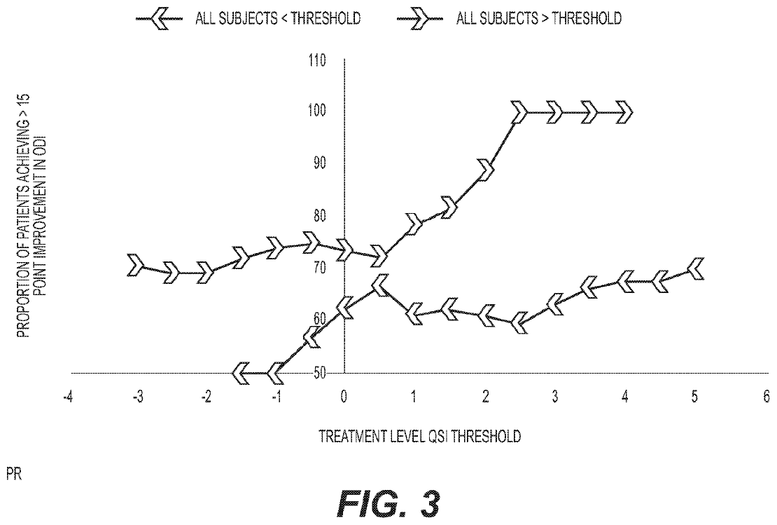

Such a graphical representation is seen in FIG. 3. In the graph you will see a first line having upward facing arrows (right facing) that will be all of the proportion of patients receiving a benefit, plotted as an increasing threshold. The second line with the arrows point downward (left facing) is a plot of the proportion of patients receiving a benefit, plotted as a decreasing threshold. As can be seen from the graph, when all patients are included 70.4% achieved a greater than 15 point improvement. The graph also shows that if the treatment is only used on patients with a QSI greater than or equal to 1, then almost 80% achieved the improvement. If the treatment is only used on patients with a QSI greater than or equal to 2.5, then 100% achieved the improvement. That is an enhanced treatment benefit. Conversely, if the treatment is only used on patients with a QSI of less than −1, then only 50% achieved the improvement. That is a reduced treatment benefit.

According to one embodiment, the methods as described herein use a pre-operative spinal trait along with patient outcomes to provide physicians with a measurable evaluation tool to assist in predicting which spinal treatment options may provide the best outcomes for their patients.

According to one embodiment the pre-operative spinal trait can be a stability metric that is calculated objectively using the method as described in the '436 patent using the ratio of translation per degree of rotation (TPDR). TPDR is calculated from the measurements of intervertebral translation and intervertebral rotation. TPDR can be calculated using any art recognized method for measuring intervertebral translation and intervertebral rotation. According to one embodiment, the method for measuring translation and rotation of the various vertebrae of the spine from images, e.g., x-ray films is described in U.S. Pat. No. 8,724,865 (hereinafter “the '865 patent”), which is incorporated herein by reference in its entirety.

In one embodiment as described herein, a method of characterizing spinal instability is disclosed using the imaging method as described in the '865 patent wherein the positions of landmarks are marked on the first film, the images are superimposed based upon adjusting the position of one image as the two images are alternately displayed until one vertebra remains in a constant position on the computer display, then repeating this process of adjusting the position of one image as the images are alternately displayed until a second vertebra is superimposed in the two images, then calculating a transformation matrix that describes the rotation and translation required to superimpose the second vertebra in the two images after first superimposing the first vertebra in the two images, and then using the transformation matrix to calculate the landmarks positions on the second film. By marking the films in this manner, the accuracy of the measurements can be maximized. In addition, difficulties in aligning the two films based upon the relative magnification and rotational position of the spine from one film to the other are addressed and minimized in the method described in the '865 patent.

The selection of at least one landmark is important only in so far as it allows consistent and reproducible measurement of translation and rotation in each patient and the asymptomatic population. Preferred landmarks for use in the disclosed methods may be chosen from the posterior superior corner of the vertebra, the centroid of the vertebra, or a specific anatomical feature found on the vertebra. The landmark that is used to measure the translation in patients must be the same landmark that is used to measure normal translation in the asymptomatic population. Accordingly a preferred landmark for measuring translation is the posterior superior corner of the vertebra. Use of this positional landmark has the added advantage that it provides a value that minimizes the effect of vertebra size. However, any chosen landmark may be corrected for vertebra size, so while posterior superior corner of the vertebra is a preferred embodiment, choice of another landmark is readily contemplated.

While the method of the '865 patent is a preferred method of measuring rotation and translation, the method as described herein may use fewer landmarks than described in the '865 patent. Variation from the '865 method are contemplated in the measurement methods as described herein and the method should only be as limited as it is defined in the instant claims.

From the two positions defined by the aligned images one can measure the intervertebral translation, the intervertebral rotation and the center of rotation. Center of rotation “COR” is well reported in the prior art and measurement methods are readily understood by the skilled artisan. As x-ray images are rarely taken at the same magnification or rotation, the center of rotation value is normalized to account for any differences in the size of the images. Any art recognized method can be used to normalize the center of rotation. According to one embodiment, the center of rotation can be normalized using the method described below with regard to the normalization of the translation value. Center of rotation can be abnormal in many ways, such as too posterior, too anterior, too cranial, too caudal or combinations of these. The COR coupled with the stability metric can provide a more complete picture to a physician regarding the nature of the instability or other abnormalities of the intervertebral motion.

Once the intervertebral translation is measured, it has to be normalized. Generally, the translation value is normalized by looking at the difference in size between a feature of the vertebra shown in the first image and the same feature of the same vertebra shown in the second image. As the method is generally carried out as a computer implemented invention, a specific vertebral dimension is measured in the first image and again in the second image. According to one embodiment, the difference in pixel size is used to normalize the translation value. According to another embodiment, the translation of the anterior-posterior width of the vertebral endplate is used to normalized the translation value. Any method for normalizing the size of the vertebral dimension can be used in the method as described herein

With the measurement of rotation and normalized translation, the TPDR can be calculated by dividing the normalized intervertebral translation by the intervertebral rotation. Along with the TPDR, the center of rotation for each position can be measured and recorded. Other metrics that can be measured and presented include measured translation, measured rotation, specific coordinates of the center of rotation such as how far above or below the endplate or how far posterior or anterior to the middle of the vertebral endplate.

Each of the measured metrics can be standardized by multiplying the measured or calculated value by a standardizing value based upon the information taken from the asymptomatic population.

The pre-treatment spinal metric can be another measureable trait, for example, standardized disc height. Standardized disc height is measured as the average disc height across the specific vertebral level. Standardized disc height is presented as a number of standard deviations from the average disc height found in radiographically normal asymptomatic volunteers at the same vertebral level. By using a standardized disc height the pre-treatment disc height may be associated with positive outcomes. Standardized disc height has been shown to be one characteristic that is predictive of outcome.

Asymptomatic population data were used to calculate average disc height as the average of 4 disc height measurements: anterior disc height measured in flexion and extension, and posterior disc height measured in flexion and extension. The average disc height calculated that way depends on the level. For example, the average disc height at the L4-L5 level is significantly greater than at the L1-L2 level. According to one embodiment, the asymptomatic data pool will continue to add new samples. According to one embodiment, the asymptomatic population data may be used to more closely align the personalized medicine aspect as the data may be subdivided into different units, based upon one more differences, for example, age, sex, weight, race, other preexisting conditions, etc. Furthermore, it may be with relation to ancestry determined from genetics.

According to another embodiment, the disc height per degree of rotation can be measured and used as the outcome predictive metric. According to yet another embodiment, the center of rotation expressed as a percent of endplate width can also be used as an outcome predictive metric.

According to another embodiment, the onset of adjacent segment disease can be evaluated. There are several potential outcomes that can be considered with respect to adjacent segment disease. One outcome is whether a medical intervention was needed to treat the adjacent segment. Another is whether there was evidence of degenerative changes. An increase in the severity of disc degeneration assessed radiographically using the Kellgren-Lawrence grading system or assessed from MRI exams using the Pfirrmann grading system would be examples.

The beneficial outcome will always be defined by the procedure or procedures that one is considering in their data development. If it's a cancer study, it may be number of years of remission, for a drug study, it may be particular symptom reduction, or lack of side effects, and for a surgical study, it might be number of post-operative complications, number of deaths during surgery, etc. According to the embodiment described herein it could be the reduction of pain, the retention of ODI improvement, etc. As will be understood, this technique can be used on any medical procedures where there exists, a beneficial outcome that can be defined, and pre-treatment outcome predictive metric.

According to another embodiment, the treatments that can be evaluated as described herein can be chosen from any medical procedure, any drug, any surgical treatment, cancer. In the spinal areas, the treatments that may be evaluated can be chosen from one or more of lumbar fusion, lumbar disc arthroplasty, cervical fusion, cervical disc arthroplasty, biologic disc regeneration treatments, disc nucleous replacements, interspinous spacers, interlaminar spacers, disc prolotherapy, disc injections, and conservative options such as traction therapy, chiropractic manipulation, watchful waiting, physical therapy.

The use of this method can provide updated support to physicians as new outcome data can be continually added to the outcome data. This expansion of the data means that the physician will be given current up to the moment information on the efficacy of the treatment method. According to one embodiment, the physician can be supplied with an app that will let them access these graphs for a variety of beneficial outcomes or treatment options and allow them to tailor their recommendations on a per patient basis.

In practice the doctor would look at a graph of, for example, patient success for Lumbar Fusion at the L2-L3 for an appropriate patient population, e.g., a general population or a female population. The doctor could then compare the threshold stability numbers of his patient against the graph to see whether the lumbar fusion has been successful for women having the same instability numbers for L2-L3. As will be understood, the information can be used to create personalized treatment plans for patients. The use of a more restricted asymptomatic population, e.g., only females or only people over 50, etc., leads to treatment plans that can be evaluated for the individual patient rather than for the population at large.

The method and system as described herein can be used in clinical diagnosis of symptomatic patients, in clinical trials of devices, technologies to treat spinal disorders, and in the continued care of individuals with spinal devices.

As will be understood from the foregoing, certain metrics will be more predictive of one treatment outcome than of a different treatment outcome. For example, one specific type of cervical disc arthroplasty may be particularly effective for a specific type of preoperative intervertebral motion abnormality, while a different type of disc arthroplasty may be better for another type of abnormality

### EXAMPLES

Development of the characteristics of an asymptomatic population may be independently developed or may be ascertained from another study of asymptomatic patients. See, for example, Sagittal plane lumbar intervertebral motion during seated flexion-extension radiographs of 658 asymptomatic nondegenerated levels, by Blake N. Staub et al., J. Neurosurg Spine, Aug. 21, 2015.

According to one embodiment, 161 asymptomatic volunteers were measured at 647 radiographically normal levels and TPDR was obtained. The measurements were made using 510K approved software, QMA®, a Medical Metrics product. The results are set forth in Table 1.

TABLE 1

| Level | Mean | UL |
|---|---|---|
| L1-L2 | 0.51 | 0.76 |
| L2-L3 | 0.56 | 0.78 |
| L3-L4 | 0.60 | 0.81 |
| L4-L5 | 0.54 | 0.47 |
| L5-S1 | 0.18 | 0.47 |

Table 1 provides the ratio of intervertebral translation (normalized to endplate width) per degree of translation (TPDR). The left-most column provides the 95% confidence interval. These data include only radiographically normal levels. The TPDR data in Table 1 cannot be used as reference data for comparison using any alternative measurement method. As will be understood by the skilled artisan, if the measurement method for translation and rotation is changed, the asymptomatic population must be re-measured using the changed method.

### Stability Metric Reliability

The method as described herein is well supported and has been compared with other known indicators of instability and has proven predictive. Standardized TPDR results, stability metrics, were calculated for different patient populations where radiographic data was available. The following correlations were found between the standardized TPDR results and other indicators of instability.

Standardized TPDR was elevated in the presence of the facet fluid sign. Facet fluid sign is considered to be one of the best currently available indicators for instability. In addition, standardized TPDR was found to be abnormally high preoperatively in a substantial proportion of lumbar fusion patients. Instability would be expected in some of those patients since instability is considered a primary indication for spine fusions. Standardized TPDR measured before treatment has also been found to be predictive of patient outcome following treatment and these types of findings can help to determine the best treatment for a patient.

### Standardized Disc Height Reliability

Asymptomatic volunteers were measured at radiographically normal levels and average disc height was obtained. The measurements were made using 510K approved software, QMA®, a Medical Metrics product.

Standardized Disc Height (SDH) is well supported by data developed by the inventor and has been used in a number of studies and has proven to be predictive of spinal treatment success. One exemplary study would be the Maverick disc study (U.S. clinical trial—a lumbar artificial disc replacement—“the Maverick is a two piece metal-on-metal design that incorporates a more posterior center of rotation”). When the results with Maverick were plotted using the invention, an enhanced treatment benefit was seen when the SDH was between −1.5 and −2.5. This indicates that the Maverick disc was particularly effective with a moderately degenerated disc.

### Example 2

Asymptomatic volunteers were radiographically evaluated for normal levels and TPDR was obtained, as described in Example 1. Symptomatic patients that had received a lumbar fusion were evaluated to determine if the benefit of the procedure was still significant at 24 months post op. In the instance the Benefit was limited to anyone having an ODI Benefit of at least 15 points of improvement at 24 months.

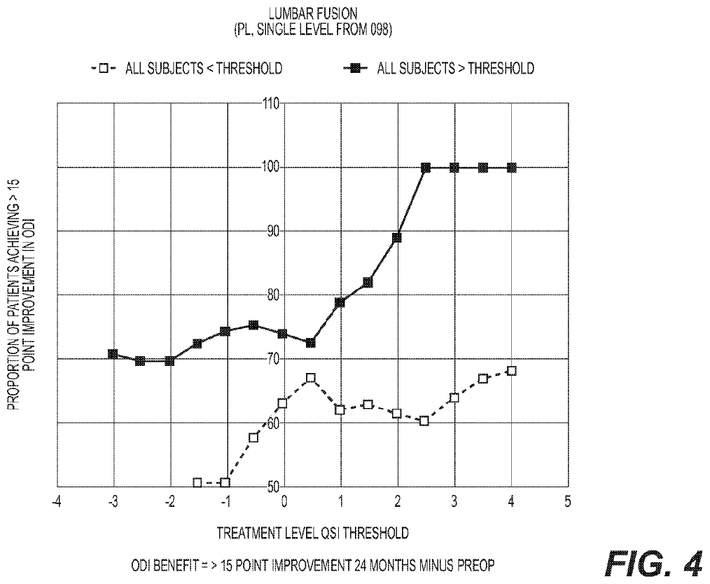

The patients who retained this benefit were plotted as a function of their initial QSI or stability number. As can be seen in FIG. 4, the graph shows that lumbar fusion was most successful on patients having a QSI greater than 0.5, and significant after a value of 2.

### Example 3

Asymptomatic volunteers were radiographically evaluated for normal levels and TPDR was obtained, as described in Example 1. Symptomatic patients that had received a lumbar disc arthoplasty were evaluated to determine if the benefit of the procedure was still significant. In the instance the Benefit was limited to anyone having an ODI Benefit of at least 15 points of improvement at 24 months.

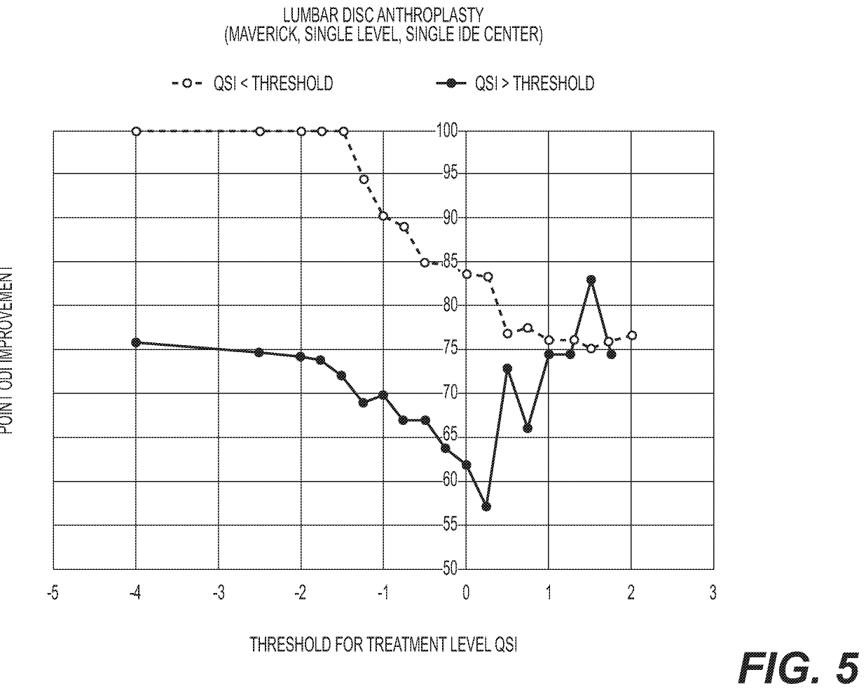

The patients who retained this benefit were plotted as a function of their initial QSI or stability number. As can be seen in FIG. 5, the graph shows that lumbar disc arthoplasty was most successful on patients having a QSI less than −1.

### Example 4

Asymptomatic volunteers were radiographically evaluated for normal levels and TPDR was obtained, as described in Example 1. Symptomatic patients that had received a lumbar disc arthoplasty were evaluated to determine if the benefit of the procedure was still significant. In this instance the Benefit was limited to anyone having an ODI Benefit of at least 15 points of improvement at 24 months.

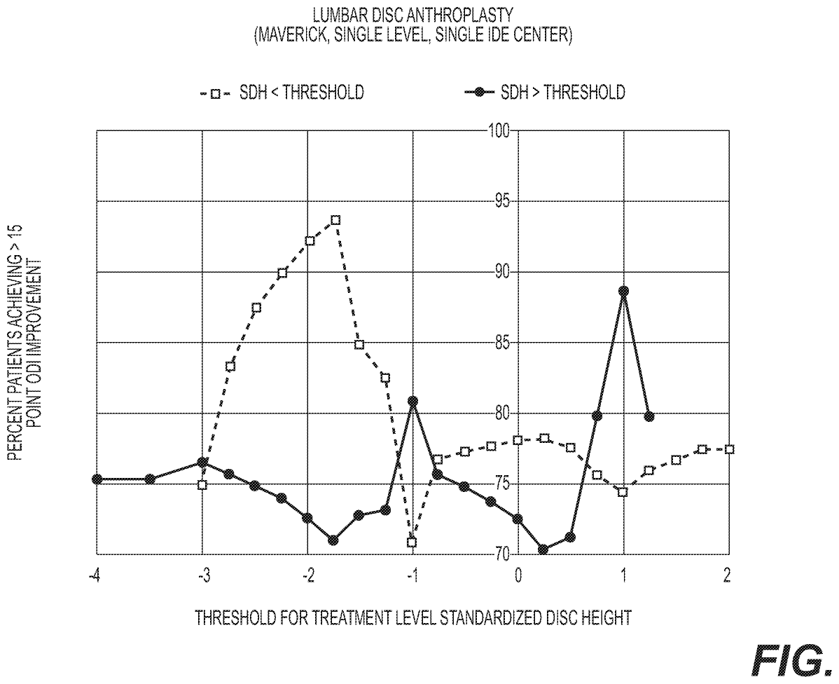

The patients who retained this benefit were plotted as a function of their initial Standard Disc Height (SDH). As can be seen in FIG. 6, the graph shows that lumbar disc arthoplasty was most successful on patients having a SDH between −2.5 and −1.5.

### Example 5

Asymptomatic volunteers were radiographically evaluated for normal levels and TPDR was obtained, as described in Example 1. Symptomatic patients that had received a cervical disc arthroplasty were evaluated to determine if the benefit of the procedure was still significant at 24 months post op. In the instance the Benefit was limited to anyone having an ODI Benefit of at least 15 points of improvement at 24 months.

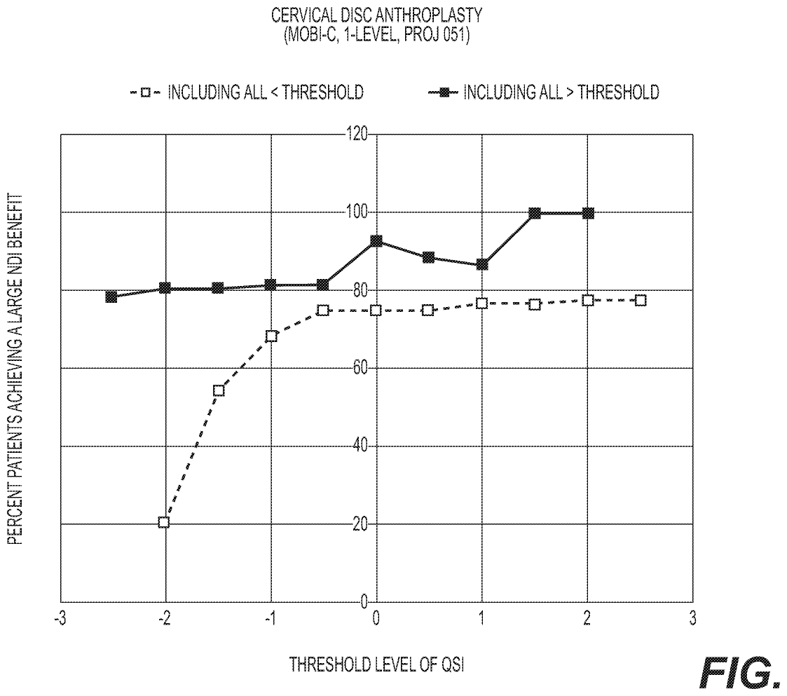

The patients who retained this benefit were plotted as a function of their initial QSI or stability number. As can be seen in FIG. 7, the graph shows that lumbar fusion was most successful on patients having a QSI between above −0.5.

Other embodiments of the present invention can include alternative variations. These and other variations and modifications will become apparent to those skilled in the art once the above disclosure is fully appreciated. It is intended that the following claims be interpreted to embrace all such variations and modifications.

What is claimed is:

1. A method for determining whether a particular treatment would provide an extended benefit or a reduced benefit to a subpopulation of patients using an outcome predictive metric comprising:
obtaining information regarding the clinical outcomes of a plurality of patients for a particular medical treatment;
determining at least one quantitative metric that has an outcome predictive correlation with the disease condition being treated;
determining the percentage of people with beneficial outcomes as a function of the at least one outcome predictive metric;
determining the baseline of beneficial outcomes by averaging the values of the quantitative metric over the least variable range of values;
calculating a range of expected benefits around the baseline beneficial outcomes;
plotting the percentage of patients with beneficial outcomes using an increasing threshold and using a decreasing threshold; and
determining if one or more sub-populations have a benefit or detriment that is outside the range around baseline of beneficial outcomes.

2. The method of claim 1, wherein the particular medical treatment is chosen from one or more of medical treatment, drug treatment, or surgical treatment.

3. The method of claim 2, wherein the treatment is an orthopedic treatment.

4. The method of claim 3, wherein the treatment is a spinal treatment and is chosen from lumbar fusion, lumbar disc arthroplasty, cervical fusion, cervical disc arthroplasty, biologic disc regeneration treatments, disc nucleous replacements, interspinous spacers, interlaminar spacers, disc prolotherapy, disc injections, traction therapy, chiropractic manipulation, watchful waiting, and physical therapy.

5. The method of claim 1, wherein the beneficial outcomes may be chosen from one or more of ODI greater than 15 at 24 months post treatment, number of years of remission, particular symptom reduction, lack of side effects, post-operative complications, number of deaths during surgery and the like.

6. The method of claim 1, wherein the range around the baseline is a 95% confidence interval for the baseline beneficial outcomes.

7. The method of claim 1, wherein the range around the baseline is defined by minimum and maximum values for the region chosen to define the baseline.

8. The method of claim 1, wherein the range around the baseline is determined using from publications describing the minimum clinically important difference (MCID) for the procedure in question, wherein the baseline value plus the MCID is the top of the range and the baseline minus the MCID is the bottom of the range.

9. The method of claim 1, wherein the outcome predictive metric is chosen from QSI and Standard Disc Height.

10. A method for determining whether a particular spinal treatment would provide an extended benefit or a detriment to a subpopulation of patients using a least one outcome predictive metric comprising:
obtaining information regarding the clinical outcomes of a plurality of patients from a particular spinal treatment;
determining at least one quantitative metric that has an outcome predictive correlation with the spinal condition being treated;
determining the percentage of people with beneficial outcomes as a function of the at least one outcome predictive metric;
determining the baseline of beneficial outcomes by averaging the values of the quantitative metric over the least variable range of values;
calculating a range of expected benefits around the baseline beneficial outcomes;
plotting the percentage of patients with beneficial outcomes using an increasing threshold and using a decreasing threshold; and
determining if one or more sub-populations have a benefit or detriment that is outside the 95% confidence interval for the baseline of beneficial outcomes.

11. The method of claim 10, wherein the beneficial outcomes may be chosen from one or more of ODI greater than 15 at 24 months post treatment, number of years of remission, particular symptom reduction, lack of side effects, post-operative complications, number of deaths during surgery and the like.

12. The method of claim 10, wherein the range around the baseline is a 95% confidence interval for the baseline beneficial outcomes.

13. The method of claim 10, wherein the range around the baseline is defined by minimum and maximum values for the region chosen to define the baseline.

14. The method of claim 10, wherein the range around the baseline is determined using from publications describing the minimum clinically important difference (MCID) for the procedure in question, wherein the baseline value plus the MCID is the top of the range and the baseline minus the MCID is the bottom of the range.

15. The method of claim 10, wherein the outcome predictive metric is chosen from QSI and Standard Disc Height.

16. The method of claim 10, wherein the range around the baseline is determined using from publications describing the minimum clinically important difference (MCID) for the procedure in question, wherein the baseline value plus the MCID is the top of the range and the baseline minus the MCID is the bottom of the range.

17. The method of claim 10, wherein the particular spinal treatment is chosen from lumbar fusion, lumbar disc arthroplasty, cervical fusion, cervical disc arthroplasty, biologic disc regeneration treatments, disc nucleous replacements, interspinous spacers, interlaminar spacers, disc prolotherapy, disc injections, and conservative options such as traction therapy, chiropractic manipulation, watchful waiting, physical therapy.

18. A method for determining whether a spinal treatment would provide an extended benefit or a detriment to a subpopulation of patients using a stability metric comprising:
obtaining an x-ray film of a first and a second vertebrae in a first position;
obtaining an x-ray film of a first and second vertebrae in a second position, determining the rotation and translation required to align the second vertebra;
measuring the degree of vertebral rotation between the first and second vertebrae in the first and second positions and calculating a TPDR;
multiplying the TPDR by a standardizing factor specific to the first and second vertebrae to generate a stability metric;
obtaining information regarding the clinical outcomes of a plurality of patients from a particular spinal treatment;
plotting the percentage of patients with beneficial outcomes as a function of the stability metric using an increasing threshold and using a decreasing threshold; and
determining if one or more sub-populations have a benefit or detriment that is outside the baseline of beneficial outcomes.

19. The method of claim 18, wherein the clinical outcomes may be chosen from ODI greater than 15 at 24 months post treatment and ODI greater than 15 at 12 months post treatment.

20. The method of claim 18, further comprising calculating a baseline of expected benefits for the total population and then determining a range around the baseline is a 95% confidence interval for the baseline beneficial outcomes.

21. The method of claim 18, further comprising calculating a baseline of expected benefits for the total population and then determining a range around the baseline is defined by minimum and maximum values for the region chosen to define the baseline.

22. The method of claim 18, further comprising calculating a baseline of expected benefits for the total population and then determining a range around the baseline is determined using from publications describing the minimum clinically important difference (MCID) for the procedure in question, wherein the baseline value plus the MCID is the top of the range and the baseline minus the MCID is the bottom of the range.

23. The method of claim 18, further comprising calculating a baseline of expected benefits for the total population and then determining a range around the baseline is determined using from publications describing the minimum clinically important difference (MCID) for the procedure in question, wherein the baseline value plus the MCID is the top of the range and the baseline minus the MCID is the bottom of the range.

24. The method of claim 18, wherein the particular spinal treatment is chosen from lumbar fusion, lumbar disc arthroplasty, cervical fusion, cervical disc arthroplasty, biologic disc regeneration treatments, disc nucleous replacements, interspinous spacers, interlaminar spacers, disc prolotherapy, disc injections, and conservative options such as traction therapy, chiropractic manipulation, watchful waiting, physical therapy.

25. A non-transitory computer readable medium encoded with a computer program that, when executed by a processor, is configured to control a method for determining whether a particular treatment would provide an extended benefit or a detriment to a subpopulation of patients using an outcome predictive metric, the method comprising:
acquiring information regarding the clinical outcomes of a plurality of patients from a particular medical treatment;
acquiring or calculating at least one quantitative metric that has an outcome predictive correlation with the disease condition being treated;
calculating the percentage of people with beneficial outcomes as a function of the at least one outcome predictive metric;
determining the baseline of beneficial outcomes by averaging the values of the quantitative metric over the least variable range of values;
calculating a range of expected benefits around the baseline beneficial outcomes;
creating a pictorial representation of the percentage of patients with beneficial outcomes using an increasing threshold and using a decreasing threshold; and
determining whether any subpopulation of patients is outside the confidence interval for the baseline benefit.
